(19) United States
(12) Patent Application Publication
(10) Pub. No.: US 2014/0072652 A1
(43) Pub. Date: Mar. 13, 2014

(54) MULTIFUNCTIONAL PRODUCE WASH

(71) Applicant: CMS Innovative Technologies, Inc., Danbury, CT (US)

(72) Inventors: David H. Creasey, Splendora, TX (US); Barry W. Cummins, Butler, KY (US)

(73) Assignee: CMS Innovative Technologies, Inc., Danbury, CT (US)

(21) Appl. No.: 14/081,301

(22) Filed: Nov. 15, 2013

Related U.S. Application Data
(63) Continuation-in-part of application No. 13/240,341, filed on Sep. 22, 2011, now abandoned.
(60) Provisional application No. 61/411,727, filed on Nov. 9, 2010.

(51) Int. Cl. A61K 33/20; A61K 31/19; A61K 31/192

(52) U.S. Cl. CPC A61K 33/20; A61K 31/192; A61K 31/19
USPC 424/600; 514/568; 514/557

(57) ABSTRACT

A produce wash is provided that reduces or eliminates the chlorine used in treating produce and inhibits the growth of microorganisms, including bacteria and mold, on pre-harvest and post-harvest produce and extends the shelf-life of the treated produce. Methods of making and using the produce wash also are provided.

### CROSS-REFERENCE TO RELATED APPLICATIONS

This application is a continuation-in-part of U.S. application Ser. No. 13/240,341, filed Sep. 22, 2011, which claims priority to U.S. Provisional Application No. 61/411,727, filed on Nov. 9, 2010, each of which is incorporated herein by reference in its entirety.

### FIELD OF THE INVENTION

This invention relates to an acidic produce wash for inhibiting the growth of mold and microorganisms that contaminate produce.

### BACKGROUND OF THE INVENTION

Edible vegetation in the human diet, such as fruit, vegetables, nuts, and crops harvested and handled in contaminated environments, carry the potential for illness and death. Well known pathogens such as Salmonella, Listeria and E. coli, as well as indicator and spoilage organisms including Staphylococcus bacteria, can be found on raw fruit, vegetables, or partially cooked foodstuffs during harvesting and processing.

The globalization of business, travel and communication brings increased attention to worldwide exchanges between communities and countries, including the potential globalization of the bacterial ecosystem. Harmful bacteria were once controlled with antibiotics, such as with penicillin in the mid-1940s, but this control no longer exists as more and more antibiotic resistant bacteria appear around the globe. For example, before 1946 about 90 percent of Staphylococcus aureus isolates in hospitals were susceptible to penicillin, but by 1952, 75 percent of isolates were penicillin-resistant. Bacterial resistance to antimicrobial agents has emerged throughout the world as one of the major threats in both human and veterinary medicine. Resistance to antibiotics and antimicrobial agents has emerged at an alarming rate because of a variety of factors, such as clustering and overcrowding, the increased use of antibiotics in animal culture and aquaculture, an increase in the number of elderly people, increased traveling, the sale of antibiotics over the counter, self-treatment with antibiotics, a lack of resources for infection control, and the inappropriate use of antibiotics.

Thus, the world population is at increased risk for acquiring antimicrobial-resistant food borne infections. Even a small increase in the prevalence of resistance in the most significant pathogenic bacteria may lead to large increases in the potential for treatment failures and other adverse outcomes, including death.

Recent media coverage has highlighted the problem associated with bacterial contamination of raw fruits such as strawberries and raspberries. In 2006, an E. coil outbreak resulted from consumption of contaminated fresh spinach. In the summer of 2008, a Salmonella outbreak linked to raw tomatoes was a reminder to take extra care with summer fruits and vegetables. Restaurateurs were asked to “Hold the Tomatoes!”

Salmonella can be transmitted to humans when fecal material from animals or humans contaminates food. Symptoms are similar to flu, but the poisoning can be fatal to young children, pregnant women and persons with weakened immune systems. Consumers cannot detect Salmonella by smell, taste or sight. One precautionary measure is to wash all produce with cold running water to remove sand and/or grit from the field, residual pesticides and bacteria. The Food Safety Institute International recommends that fruits, except bananas, should be washed, even if the peel will not be eaten.

Appropriate use of antimicrobial agents in processing foodstuffs has become necessary to avoid microbial contamination and reduce the potential for the spread of resistant organisms. Chlorine or hydrochloric acid are frequently used as bactericides and are also used universally as cleaning agents. However, one problem with compounds such as chlorine or hydrochloric acid is that they can be toxic to human beings, thereby defeating the purpose of preserving and cleaning the foodstuffs. Often chlorine or hydrochloric acid require special handling, because they can cause skin irritation and other side effects, and can even be fatal if accidentally consumed. Chlorine has other negative implications with respect to the environment, in part due to the release of chlorine gas into the environment and the requirement for special disposal methods.

Various methods for treating produce and similar compositions that reduce toxins and contamination of foodstuffs have been proposed. For example, U.S. Pat. No. 5,551,461 discloses a produce washer that resembles a dishwasher for vegetables and fruit. Produce is loaded into a basket of mesh material placed in a closeable cabinet. A washing fluid that is slightly acidic is applied, followed by a rinsing fluid, such as tap water. U.S. Pat. No. 6,506,737 discloses an antimicrobial composition for the food industry that may include sulfuric acid, sulfates, and an ammonium halide salt to provide a gel-thickened compound for cleaning and sanitizing surfaces for food preparation. The inclusion of a halide in this composition limits uses that include ingestion by man or animals and would be deleterious to machinery, plants, and other vegetation. U.S. Pat. No. 6,537,600 describes mobile systems for cleaning and drying produce in order to extend shelf life. Electrical energy and mechanical energy, such as ultrasonic energy, are employed to destroy or transform pathogens, dirt, and synthetic molecules present on the produce. U.S. Pat. No. 7,163,709 describes an ingestible disinfectant to eradicate and control pathogens on plants, animals, humans, byproducts of plants and animals, and articles infected with pathogens. The disinfectant includes sulfuric acid, water, and metallic ions, particularly copper, silver, and gold. In U.S. Pat. No. 5,989,595 and RE41,109 to Cummins, an acidic composition of matter is disclosed that is useful for destroying microorganisms that spoil food, such as fish. U.S. Pat. No. 5,989,595 and RE41,109 are each incorporated in its entirety herein by reference.

However, each of these disclosed methods has drawbacks, and a need remains for a composition that is stable over a wide range of temperatures, that minimizes eliminates the use of added chlorine, that extends the shelf-life of harvested produce, and that is effective in destroying contaminants on both pre-harvest and post-harvest produce.

### SUMMARY OF THE INVENTION

A method for producing a concentrated non-irritating produce wash for pre-harvest and post-harvest treatment of produce is provided. The method comprises

(i) selecting at least one of sulfuric acid, phosphoric acid, fumaric acid and acetic acid at a purity of at least about 98%;

(ii) combining the selected acid with water and at least one sulfate salt selected from the group consisting of sodium sulfate, ammonium sulfate, magnesium sulfate, zinc sulfate, manganese sulfate and copper sulfate to provide a first mixture;

(iii) reacting the first mixture for at least 30 minutes in a vessel capable of being pressurized and of withstanding heat and of controlling temperature of its contents to form a second mixture;

(iv) cooling the second mixture;

(v) adding at least one of a sorbate or benzoate salt to the second mixture to form the concentrated acidic produce wash.

Further provided are optional additives incorporated into the concentrated acidic produce wash may include chlorine, optionally as hydrochloric acid or a hypochlorite salt such as sodium hypochlorite.

Also provided are methods for using the produce wash to remove contaminants from produce. The methods comprise diluting the concentrated produce wash and applying diluted produce wash to pre-harvest or post-harvest produce by at least one of a spray, mist, fog, and vapor, or dipping, soaking, washing, and/or rinsing the produce in the diluted produce wash. The produce wash may also be applied to the produce as crushed ice.

### DETAILED DESCRIPTION OF THE INVENTION

A novel produce wash composition that is safe and effective in both pre-harvest and post-harvest applications and is friendly to the environment is described. The produce wash reduces the concentration of microorganisms and growth of mold on produce and extends the shelf life of harvested produce. In addition to removing microorganisms, the produce wash also removes sand and grit from the field and residual pesticides.

Certain terms used herein are defined as follows:

“Produce” refers to any edible vegetation, e.g., fruits (including vegetable fruits), herbs, leaves, stems, roots, flowers and seeds of plants, as well as edible fungi.

“Pre-harvest” refers to any time after seed germination and before the cessation of growth of Produce or of a plant product or of the life of a plant.

“Post-harvest” refers to any time after the cessation of growth of Produce or of a plant product or of the life of a plant, and includes non-food hard surfaces used in processing and preparing foodstuffs.

“Acidic component” is a combination of an acid, metallic sulfates and water reacted in a pressurizable and temperature controlled vessel, cooled and a mold preventative added to form a non-irritating low pH material partially as described in U.S. Pat. No. 8,012,511, which is incorporated in its entirety herein by reference.

“E. coli” refers to Escherichia coli, an indicator bacterial species.

“Listeria” refers to Listeria monocytogenes, a pathogen.

“Pseudomonas” refers to Pseudomonas fluorescens, a spoilage bacterial species.

“Salmonella” refers to Salmonella typhimurium, a pathogen.

“Shewanella” refers to Shewanella putrefaciens, a spoilage bacterial species.

“Staph” and “Staphylococcus” refer to Staphylococcus aureus, a pathogen.

The produce wash composition has three distinct components: (1) an acidic component that is not an irritant, (2) a sorbate and/or benzoate salt, and (3) water. These three components are mixed at room temperature to prepare the produce wash. To prepare the first component of the composition, i.e., the acidic component that is not an irritant, a pressurizable vessel that includes a means of temperature control, preferably a heating and cooling means, is selected. A preferred pressurizable vessel further is fitted with two electrodes, a cathode and an anode, to provide a direct current voltage preferably about one foot above the bottom interior surface of the vessel. The electrodes preferably are spaced about three feet apart. The acidic component is prepared in the pressurizable vessel by combining an acid, preferably in about a 1 to 2 volume ratio, with water, preferably purified water such as distilled water or deionized water or water purified by reverse osmosis, most preferably distilled water, and a sulfate salt, preferably sodium sulfate, and, optionally, one or more additional sulfate salts, preferably, copper sulfate, in the ratios listed in Table I, to form a first mixture. The acid may be sulfuric acid, phosphoric acid, fumaric acid or acetic acid, preferably sulfuric acid, with purity in the range of at least about 98%.

TABLE I
Use Levels in Milliliters per Gallon (ml/gal)

| Ratio of Sulfate Compounds | Range | Target |
|---|---|---|
| Sodium sulfate | 1.0 to 5.0 ml/gal | 3.0 ml/gal |
| Copper sulfate | 0.5 to 4.0 ml/gal | 1.0 ml/gal |

During the addition of the at least one sulfate salt, a direct current optionally may be applied to the mixture. The current may be applied in a range of about one (1) amp to about 100 amps, preferably about 1 amp to about 5 amps. The mixture reacts exothermically under pressure, typically to produce pressure of about 1 pound per square inch (psi) to about 15 psi above atmospheric pressure. The mixture also reacts to produce a temperature typically in a range of about 200° F. to about 1200° F., preferably of about 350° F. to about 400° F. The reaction duration typically is for at least about 30 minutes, but preferably for about 30 minutes to 1 hour, but may proceed for three to four hours, during which time excess hydrogen gas is removed, to form a second mixture. The temperature, time and pressure are selected and adjusted as necessary to maintain a safe chemical reaction.

After reacting, the second mixture is allowed to cool to about room temperature to form the acidic component that is not an irritant. After the cooling, a mold preventative comprising a sorbate and/or benzoate salt, preferably potassium sorbate or potassium benzoate, but which instead may be sodium sorbate or sodium benzoate, is added at less than 1 weight percent to inhibit mold growth. The resulting acidic mixture having a pH of about −1 to 2, preferably −1 to 0, is a non-irritating concentrate that is useful for destroying microorganisms and inhibiting mold growth.

This acidic component optionally may be made compatible with chlorine due to the addition of one or more sulfate salts that preferably do not include ammonium sulfate, which at high concentrations risks formation of chloramine. If chlorine is incorporated into the produce wash of the present invention, smaller amounts of chlorine can be used with greater efficacy than by using standard concentrations of chlorine alone, thereby avoiding side effects associated with chlorine use, such as health risks to workers, danger or harm to the environment, and requirements for special disposal methods.

The composition of the produce wash of the present invention, optionally including chlorine, is listed in Table II below:

TABLE II
Produce Wash

| Component | Quantity | |
|---|---|---|
| Non-irritating | 1 | gallon |
| acidic mixture | | |
| Optional | 1-5 | ml/gallon |
| chlorine | Preferably 1 | ml/gallon |
| Water | 2,000 to 3,000 | gallons |

One gallon of the non-irritating acid concentrate optionally may be combined with 1 to 5 milliliters of chlorine to make about 1 gallon of produce wash concentrate. For example, chlorine may be added as sodium hypochlorite or a solution thereof. The acidic produce wash concentrate, with or without the addition of chlorine, may easily be transported to remote farming locations and diluted on-site with a thousand gallons or more of water, which is the third component, preferably in a ratio of about 1 part of concentrated acidic produce wash to about 2,000 to 3,000 parts of water to make a diluted produce wash suitable for spraying or misting crops. The water used to dilute the produce wash can be from any source of potable water, for example, well water, municipal water sources, and desalinated drinking water. The concentration of the total of potassium sorbate and/or potassium benzoate in the diluted produce wash should be less than 1 weight percent, alternatively from 0.001 weight percent to less than 0.01 weight percent to less than 0.1 to less than 1 weight percent, of the diluted solution. The chlorine concentration optionally present in the diluted produce wash may be about 1 to 5 parts per million (ppm), which is a significant reduction compared to traditional chlorine-based washes that contain between 50 and 100 ppm chlorine at the time of application.

The produce wash may be applied in several forms when diluted with potable water, such as an aerosol, mist, vapor or fog to produce micron-sized particles that remain in suspension in the air for a period of time and act on airborne pathogens that come in contact with the composition. The produce wash may be applied to pre-harvest plants and produce as a spray or mist. The produce wash may be applied post-harvest to plants and edible plant products including, but not limited to fruits (including vegetable fruits), stems, leaves, roots, flowers and seeds. The produce wash may also be applied to edible fungi, such as mushrooms. Post-harvest treatment may be by dipping, washing, soaking, or rinsing the food product by any method that does not bruise or otherwise damage the produce. The composition can also be frozen and applied to harvested produce in the form of crushed ice. The crushed ice form of the produce wash can be used, for example, for shipping vegetables such as broccoli. Microorganisms that are killed and/or their growth inhibited by the diluted acidic produce wash include, but are not limited to, bacteria such as Salmonella typhimurium, Listeria monocytogenes, Staphylococcus aureus, Escherichia coli, Pseudomonas fluorescens and Shewannela putrefaciens and mold such as Fusarium solani . The produce wash is also effective in rinse and spray streams and chiller or ice packing, and helps to provide a safer, healthier food supply.

### EXAMPLES

### Example 1

### Chlorine Compatibility with the Acidic Component

Tests #1 and #2 in Table III measure changes in chlorine concentration over time in the acidic component prepared with electrolysis and containing 1 ppm copper and 1 or 2 ppm chlorine. These data show that there are no significant reductions in chlorine over a one hour test period. Tests #3 and #4 measure changes in chlorine concentration over time in the acidic component containing 1 ppm copper and 1 or 2 ppm chlorine, but prepared without electrolysis. These data also show that there are no significant reductions in chlorine over the one hour test period.

TABLE III
Chlorine Compatibility Tests of Acidic
Component Made with Sodium Sulfate

| | | |
|---|---|---|
| Time | Soln 1 | Soln 2 |
| (min) | (ppm) | (ppm) |

Test #1 - 1 ppm chlorine, 1 ppm copper
Acid, Water, Sodium Sulfate + electrolysis

| | | | |
|---|---|---|---|
| 0 | 1.08 | 1.08 | Starting FAC* |
| 15 | 1.09 | 1.09 | |
| 30 | 1.09 | 1.10 | |
| 45 | 1.08 | 1.08 | |
| 60 | 1.09 | 1.08 | No Reduction |

Test #2 - 2 ppm chlorine, 1 ppm copper
Acid, Water, Sodium Sulfate + electrolysis

| | | | |
|---|---|---|---|
| 0 | 1.99 | 1.99 | Starting FAC* |
| 15 | 2.00 | 2.02 | |
| 30 | 2.04 | 2.03 | |
| 45 | 2.03 | 2.02 | |
| 60 | 2.05 | 2.03 | No Reduction |

Test #3 - 1 ppm Chlorine, 1 ppm copper
Acid, Water, Sodium Sulfate - No electrolysis

| | | | |
|---|---|---|---|
| 0 | 1.09 | 1.09 | Starting FAC* |
| 15 | 1.08 | 1.09 | |
| 30 | 1.07 | 1.08 | |
| 45 | 1.09 | 1.10 | |
| 60 | 1.09 | 1.11 | No Reduction |

Test #4 - 2 ppm Chlorine, 1 ppm copper
Acid, Water, Sodium Sulfate - No electrolysis

| | | | |
|---|---|---|---|
| 0 | 2.01 | 2.00 | Starting FAC* |
| 15 | 2.02 | 2.02 | |
| 30 | 2.03 | 2.04 | |
| 45 | 2.04 | 2.02 | |
| 60 | 2.02 | 2.00 | No Reduction |

*FAC = Free Available Chlorine

These tests show no incompatibility between the low pH acidic composition made with sodium sulfate, copper sulfate and chlorine. Therefore, the composition can be effectively used in conjunction with chlorine to control microorganisms. These data also demonstrate that electrolysis does not negatively affect compatibility with chlorine.

### Example 2

### Applications for Produce Wash

The efficacy of the produce wash as an antimicrobial agent is suitable for many uses, such as those as exemplified in Table IV. The produce wash may be used on any type of produce and use is not limited to the types of produce listed in these examples.

TABLE IV
Post-Harvest Applications for Produce Wash

| Post-Harvest Application | Application Method | Water Source |
|---|---|---|
| Lettuce | In-field spray | Municipal or well |
| Tomatoes | Plant bath | Municipal or well |
| Cut bag salads | Vat baths | Municipal or well |
| Celery | In-field spray | Municipal or well |
| Spinach | In-field spray | Municipal or well |
| Broccoli | Crushed ice pack | Frozen ice blocks |
| Vegetables | Hydro chillers | Produce wash |
| | | mist |
| Sweet Bell Peppers | In-field spray | Municipal or well |

Table IV indicates the enormous commercial potential for this novel antimicrobial composition.

### Example 3

### Shelf Life Extension

Previously known standard chlorine wash contains as much as about 5,000 ppm chlorine. If more than 5,000 ppm is used, the taste of the chlorine becomes noticeable, which is undesirable for the consumer. In addition, at 5,000 ppm chlorine, a standard chlorine wash is microstatic, i.e., the standard chlorine wash inhibits growth of microorganisms, but does not kill them. Eventually, the microorganisms will begin to reproduce, leading to produce spoilage.

Reduced TBC (total bacteria count) correlates with expanded shelf life of produce. As shown in Table V, pre-harvest application of the produce wash eliminated bacteria and other contaminants on produce at a very low concentration of chlorine (Cl). As a result, shelf life was improved by 50-60% (3-5 days) compared with produce treated with a standard chlorine produce wash.

TABLE V
Shelf Life in Days

| | Produce wash | Chlorine wash |
|---|---|---|
| Pre-Harvest Application | 2 ppm Cl | ≧5000 ppm Cl |
| Lettuce | 8-12 days | 5-7 days |

### Example 4

### Romaine Heart Lettuce Samples —Bacteria Study 33 Days Post-Harvest

Lettuce samples were submitted for analysis by standard test methodology from the Bacteriological Analytical Manual (BAM), U.S. Food and Drug Administration, 11^{th} edition. Results are shown in Table VI. Colony forming units per gram (cfu/g) represent counts of live bacteria or other contaminants after 33 days.

TABLE VI
Romaine Heart Lettuce Samples - Bacteria Study 33 days Post-Harvest

| SAMPLE ID. | RH-1 | TRH-1 |
|---|---|---|
| Aerobic Heterophilic (TPC), cfu/g | 170,000 | 10,000 |
| Fecal Coliforms, cfu/g | 50 | 13 |
| E. coli, cfu/g | 13 | None Detected |
| Staphylococcus, cfu/g | None Detected | None Detected |
| Salmonella/Shigella, cfu/g | None Detected | None Detected |

RH-1 is Romaine Heart Lettuce treated with a standard chlorine wash. TRH-1 is Romaine Heart Lettuce treated with produce wash. These data show that the produce wash caused significant reductions in contaminants, i.e., a 17-fold reduction in Aerobic Heterophilic total plate count (TPC), colony forming units per gram (cfu/g) compared with a standard chlorine wash.

### Example 5

### Green Leaf Lettuce Samples 33 Days Post-Harvest

Chopped Green Leaf Lettuce (CHL) was treated with standard chlorine wash. Chopped and Bagged (T-bag) Green Leaf Lettuce was treated with produce wash. The effect of these treatments on bacterial counts are shown in Table VII. Produce wash caused significant reductions in all contaminants, with a 91-fold reduction in Aerobic Heterophilic (TPC), cfu/g when compared with treatment with a standard chlorine wash.

TABLE VII
Green Leaf Lettuce Samples 33 days Post-Harvest

| SAMPLE ID. | CGL-1 | T-Bag |
|---|---|---|
| Aerobic Heterophilic (TPC), | 1,280,000 | 14,000 |
| cfu/g | | |
| Fecal Coliforms, cfu/g | ≧1600 | None Detected |
| E. coli, cfu/g | 53 | None Detected |
| Staphylococcus, cfu/g | 80 | None Detected |
| Salmonella/Shigella, cfu/g | 4 | None Detected |

### Example 6

### Iceberg Lettuce Samples 33 Days Post-Harvest

Iceberg Lettuce was treated with standard chlorine wash (C-IB1) or with produce wash (T-IB1) 33 days after harvest. Table VIII shows that produce wash caused significant reductions in all contaminants, with a 15-fold reduction in Aerobic Heterophilic (TPC), cfu/g when compared to iceberg lettuce washed with a standard chlorine wash.

TABLE VIII
Iceberg Lettuce Samples 33 days Post-Harvest

| SAMPLE ID. | C-IBI | T-IB1 |
|---|---|---|
| Aerobic Heterophilic (TPC), cfu/g | 600,000 | 40,000 |
| Fecal Coliforms, cfu/g | ≧1600 | 13 |
| E. coli, cfu/g | 93 | 2 |
| Staphylococcus, cfu/g | 23 | 2 |
| Salmonella/Shigella, cfu/g | None Detected | None Detected |

### Example 7

### Inhibition of Mold by Dilute Acidic Produce Wash

Mold growth is inhibited by the dilute acidic produce wash of the invention as shown in Table IX. Cells from a stock culture of Fusarium solani ATCC #36031 were transferred from a cell-laden swab onto the surface of Potato Dextrose Agar culture plates. A 0.1 mL sample of dilute acidic produce wash comprising <1% potassium benzoate or <1% potassium sorbate or neither as a control was applied onto the center surface or into an 8 mm bore hole of an inoculated culture plate and incubated for 6 days at 37±2° C. Table IX shows that the dilute acidic produce wash containing either potassium benzoate or potassium sorbate resulted in a measured zone of growth inhibition (“Zone”) compared to no growth detectable inhibition in the control.

TABLE IX
Inhibition of Mold by Dilute Acidic Produce Wash

| SAMPLE ID. | SURFACE SAMPLE | BORE SAMPLE |
|---|---|---|
| Potassium | 27 mm Zone | 37 mm Zone |
| benzoate | | |
| Potassium sorbate | 25.7 mm Zone | 15.9 mm Zone |
| No inhibitor | No Zone | No Zone |

Although the invention is illustrated and described herein with reference to specific embodiments, the invention is not intended to be limited to the details shown. Various modifications may be made in the details within the scope and range of equivalents of the claims and without departing from the invention.

We claim:

1. A method of producing a concentrated produce wash for pre-harvest and post-harvest treatment of produce comprising
(i) selecting at least one of sulfuric acid, phosphoric acid, fumaric acid and acetic acid having a purity of at least about 98%;
(ii) combining the selected acid of (i) with water and at least one sulfate salt selected from sodium sulfate or ammonium sulfate or magnesium sulfate to provide a first mixture;
(iii) reacting the first mixture of (ii) in a vessel capable of being pressurized and of withstanding heat to form a second mixture;
(iv) cooling the second mixture;
(v) adding to the cooled second mixture a solution of at least one of a sorbate or benzoate salt as a mold inhibitor to form a concentrated acidic produce wash.

2. The method of claim 1, wherein the ratio of sulfate to water in step (ii) is about 1 to about 5 pounds of sulfate salt per gallon of water.

3. The method of claim 1, further comprising mixing at least one additional sulfate salt selected from the group consisting of zinc sulfate, manganese sulfate and copper sulfate with the sulfate salt of step (ii).

4. The method of claim 1, wherein the reacting of step (iii) takes place at a temperature of about 200° F. to about 1200° F.

5. The method of claim 1, wherein the reacting of step (iii) takes place at a pressure of about 3 psi to about 15 psi.

6. The method of claim 1, further comprising adding after cooling a stabilizer that comprises about 10 weight percent of the total weight of the first mixture in step (ii) to provide a stable, non-irritating acidic component having a pH of about −1 to about 0, wherein the stabilizer comprises an aqueous solution of sodium sulfate at a concentration of about 1 to 5 pounds per gallon.

7. The method of claim 1, wherein the reacting of step (iii) is for about 30 minutes to about 1 hour.

8. The method of claim 1, wherein the reacting of step (iii) is for about 1 hour to about 4 hours.

9. The method of claim 1, further comprising applying direct current voltage to the acid while combining the sulfate with the acid in step (ii).

10. The produce wash of claim 9, wherein the direct current is about 1 amp to about 100 amps.

11. The method of claim 1, further comprising diluting the concentrated acidic produce wash with potable water in a ratio of about 1 part concentrated acidic produce wash to about 2000 to 3000 parts potable water to yield a dilute acidic produce wash suitable for pre-harvest and post-harvest treatment of produce.

12. The method of claim 11, wherein the dilute acidic produce wash has a chlorine concentration of about 1 ppm to about 5 ppm.

13. A method for removing contaminants and at least one of killing a microorganism or inhibiting the growth of a microorganism on pre-harvest or post-harvest produce comprising applying the dilute produce wash of claim 11 to pre-harvest or post-harvest produce.

14. The method of claim 13, wherein the dilute produce wash is applied as at least one of a spray, mist, fog, or vapor in the field where the produce is grown.

15. The method of claim 13, wherein the dilute produce wash is applied by at least one of dipping, soaking, washing, and rinsing the produce in the dilute produce wash.

16. The method of claim 13, wherein the produce wash is frozen to form an ice, crushed, and applied to post-harvest produce.

17. The method of claim 13, wherein the treated produce has an extended shelf life of 1 to 5 days longer than produce that is not treated with the dilute produce wash.

18. A method of inhibiting growth of at least one of a known pathogenic, indicator and spoilage bacteria or a mold during pre-harvest and post-harvest treatment of produce comprising applying the dilute produce wash of claim 11 to pre-harvest or post-harvest produce.

19. The method of claim 19, wherein the known pathogenic, indicator and spoilage bacteria are selected from the group consisting of Salmonella typhimurium, Listeria monocytogenes, Staphylococcus aureus, Escherichia coil, Pseudomonas fluorescens and Shewannela putrefaciens.

20. A concentrated acidic produce wash comprising the reaction product of at least one of sulfuric acid, phosphoric acid, fumaric acid or acetic acid having a purity of at least about 98%; water; a sulfate salt selected from at least one of sodium sulfate or ammonium sulfate or magnesium sulfate, and further comprising at least one of a sorbate salt or a benzoate salt.

21. The concentrated acidic produce wash of claim 21, diluted in water at a ratio of one part concentrated acidic produce wash to 2,000 to 3,000 parts of potable water to form a diluted acidic produce wash.

22. The concentrated acidic produce wash of claim 21, wherein the sulfate salt is sodium sulfate.

23. The concentrated acidic produce wash of claim 23, further comprising copper sulfate.

24. The concentrated acidic produce wash of claim 21, wherein the sorbate and benzoate salts are potassium sorbate and potassium benzoate, respectively, and the concentration of the total of sorbate salt and benzoate salt is less than 1 weight percent of the diluted acidic produce wash.
